(19) United States
(12) Patent Application Publication
(10) Pub. No.: US 2008/0124281 A1
(43) Pub. Date: May 29, 2008

(54) NANOTUBULAR PROBES AS ULTRASENSITIVE MR CONTRAST AGENT

(75) Inventors: Jinming Gao, Dallas, TX (US); Heather L. Hillebrenner, Dallas, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/564,742

(22) Filed: Nov. 29, 2006

(51) Int. Cl. A61B 5/055

(52) U.S. Cl. 424/9.32; 977/788; 977/811; 977/930

(57) ABSTRACT

The present invention includes compositions, methods and methods for using MRI contrast agent that include a generally nanotubular carrier and an MRI contrast agent disposed within the carrier.

### TECHNICAL FIELD OF THE INVENTION

The present invention relates in general to the field of contrast agents, and more particularly, to compositions and methods for making and using nanotubular carriers for MRI contrast agents.

### BACKGROUND OF THE INVENTION

Without limiting the scope of the invention, its background is described in connection with molecular imaging.

Molecular imaging is becoming an important discipline that investigates disease-specific molecular information through diagnostic imaging methods (Weissleder, et al., JAMA 2005, 293, 855). Among various imaging modalities, magnetic resonance imaging (MRI) provides superb in vivo imaging capability with high resolution (<1 mm), excellent soft tissue contrast, and sensitivity to blood flow. The primary limitation of MRI has been its lower sensitivity for the detection of targeted agents over other imaging modalities (e.g., nuclear imaging).

### SUMMARY OF THE INVENTION

The present invention addresses a major limitation in the molecular imaging of specific pathological markers by MRI is the low sensitivity of detection of the contrast agents. For example, the Gd-DTPA complex has millimolar (mM) detection limit that is too high for detecting specific molecular markers under physiological conditions. In this invention, we demonstrate the feasibility of achieving picomolar (10⁻¹² M) detection limit by MRI through the SPIO-loaded nano test tubes. Current T2-based MRI contrast agents are Fe₃O₄ nanoparticles encapsulated in the hydrophilic dextran matrix. The contrast agents are variable in size and distribution, and the detection sensitivity is limited.

The compositions and methods of the present invention can be used to encapsulate a large quantity of SPIO particles to enhance MR signal. The nanotubes used herein provide a larger surface area for attaching targeting ligands for better targeting to specific pathological markers. Disclosed herein are novel compositions, methods of making and methods of using nanotubes loaded with MR contrast agents, which may also be functionalized. In synthesized from anodic alumina templates with tube dimensions ≦100 nm in diameter and ≦500 nm in length. The nanotubes are filled with superparamagnetic iron oxide nanoparticles (SPIO) to achieve picomolar detection limit by magnetic resonance imaging (MRI). The surface of the nanotubes can be functionalized with targeting ligands for molecular imaging applications in cancer or other pathological conditions.

The nanotubular design of the present invention has the following potential advantages: (1) precise control of particle size and shape (e.g., tube length and diameter); (2) high SPIO payload capacity; (3) differential inner and outer surface functionalization; (4) prolonged blood circulation time through aligned nanotube orientation with blood flow direction.

More particularly, the present invention includes an MRI contrast agent that includes a generally nanotubular carrier; and an MRI contrast agent disposed within the carrier. The carrier can be biocompatible, biodegradable or both and may include one or two open ends. If either of the carrier ends are open, the carrier may be capped at one or both ends. The carrier may be made from a biodegradable polymer selected from polysaccharides, cellulose, chitosan, carboxymethylated cellulose, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides, polyalkyl(cyano)acrylates, poly(trimethylene carbonate) and copolymers, poly(ε-caprolactone) homopolymers and copolymers, polyhydroxybutyrate and polyhydroxyvalerate, poly(ester)urethanes and copolymers, polymethyl-methacrylate and combinations thereof. Alternatively, the carrier may be made from polyglutamic or polyaspartic acid derivatives and their copolymers with other amino-acids. Examples of contrast agents that can be loaded into the nanotube carries include superparamagnetic iron oxide nanoparticles.

Other examples of MRI contrast agents for use with the present invention include any superparamagnetic iron oxide selected from the compositions of MFe₂O₄, wherein M=Fe, Co, Ni, Zn, Mg, Mn divalent metal ions. In another example, the contrast agent is a hydrophilic, a hydrophobic, a polar, a non-polar, a non-ionic, an anionic or a cationic MRI contrast agent or combinations thereof. The carrier may be made from a silica tubule and the contrast agent comprises superparamagnetic iron oxide nanoparticles and the contrast agent is detectable at a concentration of less that 10 pM. The carrier may also be functionalized, e.g., functionalized and a targeting ligand bound to the carrier. The carrier may be functionalized with, e.g., using amines, carboxylic acids, thiols, aldehydes and combinations thereof. The carrier may also be functionalized with a cross-linking agent selected from glutaraldehydes, diamines, and disulfides and combinations thereof. A targeting ligand may be any agent with at least partial target selectivity, e.g., the targeting ligand may be aptamers, peptides, small organic molecules (e.g., folic acid), antibodies, proteins, oligosaccharides and combinations thereof. The carrier may be a biocompatible inorganic tubule selected from iron oxide, titanium dioxide, silicon oxide or combinations thereof.

The present invention also includes a method for making an MRI contrast agent by forming a nanotubular carrier and loading the nanotubular carrier with an MRI contrast agent. The carrier can be biocompatible, biodegradable or both and may include one or two open ends. If either of the carrier ends are open, the carrier may be capped at one or both ends. The carrier may be made from a biodegradable polymer selected from polysaccharides, cellulose, chitosan, carboxymethylated cellulose, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides, polyalkyl(cyano)acrylates, poly(trimethylene carbonate) and copolymers, poly(ε-caprolactone) homopolymers and copolymers, polyhydroxybutyrate and polyhydroxyvalerate, poly(ester)urethanes and copolymers, polymethyl-methacrylate and combinations thereof. Alternatively, the carrier may be made from polyglutamic or polyaspartic acid derivatives and their copolymers with other amino-acids. Examples of contrast agents that can be loaded into the nanotube carries include superparamagnetic iron oxide nanoparticles.

The present invention also include a method for assessing tissue in a patient using a magnetic resonance imaging (MRI) apparatus, the method includes injecting into the patient a generally tubular nanocarrier comprising an MRI contrast agent within the nanocarrier.

An MRI contrast agent may include a nanotubular carrier, an MRI contrast agent loaded into the carrier and a targeting ligand bound to the carrier. The method for making an MRI contrast agent by forming a nanotubular carrier; loading the nanotubular carrier with an MRI contrast agent; and functionalizing the surface of the carrier a targeting ligand.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic synthesis of SPIO-loaded nano test tubes;

FIGS. 2A to 2C show SEM (2A and 2B) and TEM (2C) images. FIG. 2A shows Alumina template cross-section, scale bar=300 nm, FIG. 2B shows the template-free silica nano test tubes, scale bar=1 μm and FIG. 3C shows SPIO-loaded silica nano test tubes, scale bar=500 nm, inset scale bar=200 nm;

FIG. 3 shows T2-weighted MR images of SPIO-NTTs vs. unloaded NTTs. The concentration of NTTs per sample are listed below the corresponding image;

FIG. 4 is a graph of the MRI intensity as a function of SPIO-NTT concentrations in 1% agarose gel by T2-w imaging using a spin-echo sequence (TE=9, 20 and 65 ms). The control sample represents empty NTTs without SPIO loading with TE=65 ms;

FIG. 5 is a comparison of the payload capacity between a micelle and a nano test tube;

FIG. 6 is a schematic of one example of a method of functionalizing the nanotubes, in this embodiment using a cRGD-SPIO-NTC synthesis.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, Superparamagnetic Iron Oxide (SPIO) agents are composed of iron oxide nanocrystals with the general formula Fe₂³⁺O₃M²⁺O, where M²⁺ is a divalent metal ion such as iron, manganese, nickel, cobalt, zinc, or magnesium. When M²⁺ is ferrous iron (Fe²⁺), SPIO becomes magnetite (Fe₃O₄). In the absence of an external magnetic field, the magnetic domains inside SPIO are randomly oriented with no net magnetic field. An external magnetic field can cause the magnetic dipoles of the magnetic domains to reorient, leading to dramatically increased magnetic moments, and significantly shortened relaxations in both T₁ and T₂/T₂* relaxation processes. SPIO nanoparticles are considered T₂-negative contrast agents with high T₁ and T₂ relaxivities.

A wide variety of agents and methods that are well-known in the art may be used to functionalize the nanotube carriers for use with the present invention. For example, methods to couple ligand to tube surface includes the use of commercially available silanes with functional groups such as amines, carboxylic acids, thiols, and aldehydes can be used to add functionality to the surface of the silica nanotubes as well as other oxides. These functional groups can then be used to couple the ligand to the tube surface. Examples of techniques include those taught by Mitchell, D. T.; et al., Smart nanotubes for bioseparations and biocatalysis. Journal of the American Chemical Society 2002, 124, (40), 11864-11865; and Martin, C. R. and Kohli, P., The emerging field of nanotube biotechnology. Nature Reviews Drug Discovery 2003, 2, (1), 29-37, relevant portions incorporated herein by reference.

Examples compounds and methods for cross-linking the tube and a ligand may include, e.g., glutaraldehydes, diamines, and disulfides and combinations thereof. Functional groups for use with the present invention may include those that are target and/or organ specific. Examples of targeting ligands include, e.g., aptamers, peptides, small organic molecules, antibodies, proteins, folic acid, oligopeptides, oligosaccharides.

The nanotubes may be made from a wide variety of materials, e.g., organic, inorganic, polymeric, biodegradable, biocompatible and combinations thereof. Non-limiting examples of inorganic materials to make the nanotube carriers of the present invention include iron oxide, silicon oxide, titanium oxide and the like. Examples of biodegradable monomers formed into a nanotubular carrier include polysaccharides, cellulose, chitosan, carboxymethylated cellulose, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides, polyalkyl(cyano)acrylates, poly(trimethylene carbonate) and copolymers, poly(ε-caprolactone) homopolymers and copolymers, polyhydroxybutyrate and polyhydroxyvalerate, poly(ester)urethanes and copolymers, polymethyl-methacrylate and combinations thereof. The carrier may even include or made from polyglutamic or polyaspartic acid derivatives and their copolymers with other amino-acids.

Breast cancer is currently the leading cause of cancer death in women.⁵ Generally, surgical resection remains the mainstay for breast cancer treatment, because in many cases the onset of the disease goes undetected until symptoms appear and it is too late for less invasive intervention. Conventional mammography has been the first line of defense as a diagnostic tool for the past 20 years but tends to give false positives in the range of 60-80%.⁶ For breast tumor growth, local invasiveness, and cancer metastasis, angiogenesis plays a key role. Currently, the most accepted method used to assess tumor-induced angiogenesis is the determination of intratumoral microvessel density (MVD) by tissue biopsy in areas of the most active neovascularization.^{7, 8} The invasiveness of this technique is an obvious limitation since periodic assessment of anti-angiogenic efficacy in the same patient may be required.

To circumvent this, a non-invasive, clinical MRI technique called dynamic contrast enhancement MRI (DCE-MRI) has been used to assess tumor angiogenesis.^{6, 9-14} Although DCE-MRI has been implemented clinically, vascular assessment is not specific or precise.¹⁰ It has been shown that not only malignant, but benign lesions along with fibroadenoma, mastitis, and normal breast tissue, depending on the menstrual cycle, all show enhancement after the administration of contrast agents.^{10, 12} Duerk and coworkers have shown that artifacts in the tissue portion of baseline T1 images affect the accuracy of K^{trans} measurement.⁹ Because of these limitations, successful development of cancer-specific and ultra-sensitive contrast agents will have a major impact on the early detection of breast cancer as well as non-invasive monitoring of therapeutic outcome of drug therapy.

Described herein is the development of a novel MRI contrast agent with picomolar (pM) detection sensitivity on a per particle basis. Silica nano test tubes (dimensions 100 nm×500 nm) were synthesized using an alumina template and further loaded with SPIO particles (11 nm in diameter). T2-weighted MR imaging was performed to examine the SPIO-loaded NTT sample at different concentrations in a 1% agarose gel and compared to empty NTTs. This unique nanotubular design has allowed us to achieve an ultra-sensitive detection limit at 1.1 pM (50% MRI intensity, TE=65 ms), which may open up many exciting opportunities in molecular imaging applications.

The development of superparamagnetic iron oxide (SPIO)-loaded polymeric micelles is an effective strategy to enhance MRI sensitivity of detection. MRI detection limit at nanomolar (˜nM) concentrations of micelles were achieved as a result of the high loading of SPIO inside the micelles. The development of a nanotubular design of MR imaging probes to further increase the MR sensitivity of detection based on increased SPIO loading and asymmetrical tubular design down to the picomole level.

Advantages of nanotubes over spherical particles include, e.g., higher payload capacity; differential inner and outer surface functionalization; the nanotubes can self-orient with fluid flow direction (i.e. blood flow); prolonged blood circulation time; and/or the templates are tunable to, e.g., material, tube length and diameter can be controlled.

Briefly, silica nano test tubes (NTTs) were synthesized from home-grown alumina templates (FIG. 1) (see, e.g., Gasparac, R.; Kohli, P.; Paulino, M. O. M.; Trofin, L.; Martin, C. R., Template synthesis of nano test tubes. Nano Letters 2004, 4, (3), 513-516, relevant portions incorporated herein by reference). The NTT dimensions in these proof-of-principle experiments were 100 nm in diameter and 500 nm in length. The inner pores of the tubes were loaded with 11 nm SPIO particles. SPIO-loaded NTT samples were prepared by suspending the tubes in 1% agarose gel. All MRI studies were conducted using a Litz coil (diameter 4 cm, length 5 cm, DOTY Scientific INC, NC) on a 4.7 T horizontal scanner (Varian, Calif., USA) at room temperature (˜20° C.). T2-weighted imaging of the phantom samples were collected using a spin-echo pulse sequence with a repetition time of 6.0 s and varying echo times of 9, 20, and 65 ms. The MRI images were processed using the Image J software (a freeware from the NIH).

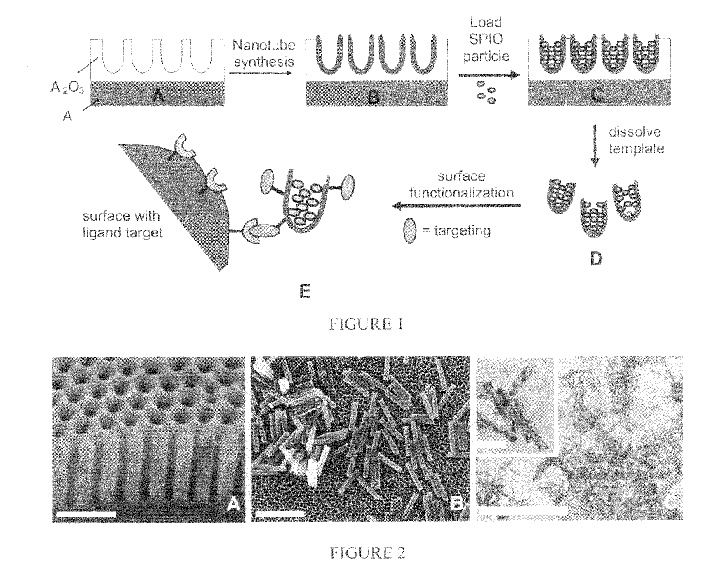

SPIO-loaded NTTs were produced and loaded as illustrated in FIGS. 2A to 2C. First, layer-by-layer deposition was used to synthesize silica NTTs. Second, SPIO particles were loaded in the NTTs when the NTTs were membrane bound. Upon membrane dissolution, the NTTs were collected and examined by TEM to verify SPIO loading (FIG. 2C). Due to the hydrophobic surface coating of the SPIO particles, the loaded SPIO did not leak out of the NTTs even after suspension in water for 7 days as determined by TEM examination.

T2-weighted imaging was carried out to evaluate the MR sensitivity of detection. Images were processed and mean gray value intensity was measured and normalized to the agarose gel control without NTTs (FIG. 3). SPIO-free NTT samples were used as a control. For all the samples, MR intensity decreased when the SPIO-loaded NTT concentration was increased. Moreover, increasing TE time also led to a considerable decrease of MR intensity at low NTT concentration. The sensitivity limits were 1.1 pM, 4.3 pM and 8.6 pM for TE values at 65, 20 and 9 ms, respectively. The sensitivity limit of detection is defined as the NTT concentration where the MR intensity is decreased to 50% of the control sample. These results suggest an approximately 1000 fold increase in sensitivity over previously published micellar systems (5 nM) (Gao, et al., Adv. Mat. 2005, 17, 1949,). The dramatic increase in sensitivity serves to expand the use of MR probes in imaging specific markers in molecular imaging applications.

The feasibility of SPIO-loaded NTTs as a novel ultrasensitive platform with a picomolar (pM) detection limit. For proof-of-concept studies, tubes of dimension 100 nm×500 nm were used. Future studies are in progress to decrease the size of NTTs (e.g., 50 nm×200 nm) and functionalize the NTT surface with cell targeting ligands for molecular imaging applications in cancer.

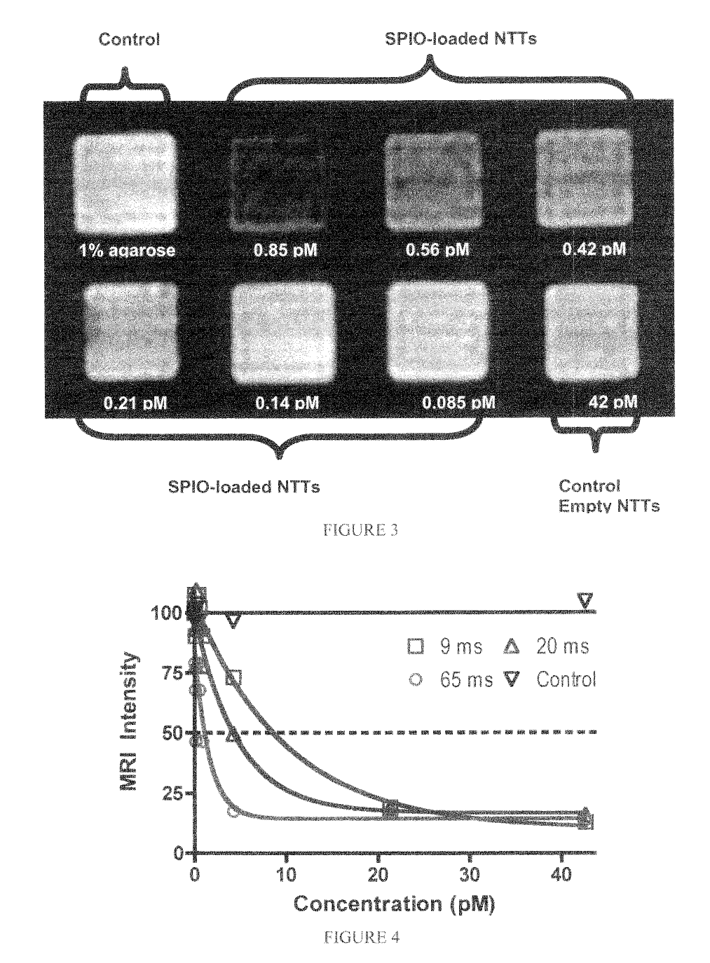

FIG. 4 is a graph that compares the MRI intensity as a function of SPIO-NTT concentrations in 1% agarose gel by T2-w imaging using a spin-echo sequence (TE=9, 20 and 65 ms). The control sample represents empty NTTs without SPIO loading with TE=65 ms and shows the increase in sensitivity of the MRI intensity at in the picomolar range.

As a new probe design for the early detection of cancer, functionalized silica nanotubular capsules (NTCs) may be used in conjunction with contrast agents for magnetic resonance imaging (MRI). NTCs are made by template synthesis of silica nano test tubes, followed by the loading of superparamagnetic iron oxide (SPIO) particles (loaded tubes closed at one end, see, e.g., FIG. 2C, insert). These test tubes are then capped to ensure SPIO particles remain in the inner cavity, forming silica NTCs. The loading of NTCs with SPIO particles will achieve a highly sensitive and specific detection of angiogenic tumor vessels by MRI. The cylindrical shape of the NTCs will increase the magnetization characteristics and improve the sensitivity of cancer detection. Moreover, encoding of NTC outer surface with cancer targeting peptide, cyclic RGD, will allow for cancer-specific imaging of tumor angiogenesis.

Development and characterization of cRGD-encoded, SPIO-loaded silica nanotubular capsules (cRGD-SPIO-NTCs) for in vitro studies. Silica nano test tubes can be template-synthesized using well established methods,^{1, 2} followed by loading of the inner cavity with SPIO particles, and (optionally) capping.^{3, 4} Membrane dissolution releases the NTCs and expose the outer surface hydroxyl groups, which will be further functionalized with cyclic pentapeptide c(Arg-Gly-Asp-D-Phe-Lys), cRGD. cRGD-SPIO-NTCs will be incubated with αᵥβ₃-overexpressing SLK cells in the presence and absence of αᵥβ₃ blocking antibody (LM609). The resulting cell suspension can be imaged by MRI and also compared to cells incubated with SPIO-NTCs without cRGD encoding.

Evaluation of in vivo imaging efficacy of cRGD-SPIO-NTCs in mice bearing breast tumor xenografts by MRI. cRGD-SPIO-NTCs can be injected i.v. in athymic nude mice bearing MCF-7 and MB-MDA-231 breast tumor xenografts. Tumor imaging specificity can be evaluated by comparing the imaging efficacy of cRGD-SPIO-NTCs with SPIO-NTCs without cRGD modification, and cRGD-SPIO-NTCs co-injected with high concentrations of free cRGD peptide. NTC distribution in other organs and tissues will also be evaluated and compared. After MR imaging, breast tumor xenografts will be removed for histology analysis. Microvascular density will be measured and correlated with MRI imaging data.

SPIO as T2 contrast agents. Over the past decade the most extensively studied MR contrast agents have been superparamagnetic iron oxide (SPIO) nanoparticles. The use of SPIO as MRI contrast agents for liver and spleen diagnosis is now a well-established area of pharmaceutical development. Several SPIO formulas are commercially available or in clinical trials, including Feridex®, Endorem™, GastroMARK®, Lumirem®, Sinerem®, Resovist®.

Unlike the low molecular weight, paramagnetic metal chelates such as Gd-DTPA (T1 contrast agent), SPIO nanoparticles are considered T2-negative contrast agents with substantially higher T2 and T1 relaxivity compared to T1 agents.¹⁵ SPIO agents are composed of iron oxide nanocrystals with the general formula Fe₂³⁺O₃M²⁺O, where M²⁺ is a divalent metal ion such as iron, manganese, nickel, cobalt, or magnesium. When M²⁺ is ferrous iron (Fe²⁺), SPIO becomes magnetite. In the absence of an external magnetic field, the magnetic domains inside SPIO are randomly oriented with no net magnetic field. An external magnetic field can cause the magnetic dipoles of the magnetic domains to reorient, leading to dramatically increased magnetic moments, and significantly shortened relaxations in both T1 and T2/T2* relaxation processes.¹⁵ It is worth noting that the T2 relaxivities of micelles notably depend on SPIO clustering, SPIO diameter, and loading density. Therefore, the more SPIO clustering, led to greater T2 relaxivities resulting in a more sensitive contrast agent. Previously, Gao and coworkers have displayed promising results using spherical micelle carriers for the SPIO particles¹⁶. However, a new probe design using template synthesized silica nano test tubes was developed to increase the sensitivity to further enhance the MRI sensitivity and cancer specificity for breast tumor detection.

Targeting tumor vasculature via cRGD ligand. As a tumor begins to grow it releases chemicals that promote the growth of new capillaries to supply it with more blood and nutrients. This process is referred to as tumor vascularization or angiogenesis. During angiogenesis a unique biomarker receptor (αᵥβ₃ integrin) is over-expressed on the luminal surface of endothelial cells.¹⁷ Although all endothelial cells use integrins to attach to extraluminal submatrix, this αᵥβ₃ integrin is specific for differentiation of newly formed capillaries from their mature counterparts.¹⁸ Vascular targeting via αᵥβ₃-dependent mechanisms during the early stages of angiogenesis can be accomplished by detection with MRI combined with contrast agents that specifically target αᵥβ₃₃ integrins.

Recently, the crystal structures of the extracellular domains of αᵥβ₃ and αᵥβ₃/c(-RGDfV-) complex have been determined by Xiong et al.^{19, 20} These structures provide molecular insights and better understanding of αᵥβ₃ receptor-ligand interactions and establish the structural basis for future development of more potent and specific αᵥβ₃-binding ligands. Cheresh and coworkers reported the success of αᵥβ₃-targeted gene therapy of cancer with cationic polymerized lipid-based nanoparticles.²¹ These data provide useful precedence for introducing αᵥβ₃-targeted ligands such as cyclic (Arg-Gly-Asp-D-Phe-Lys) (cRGD) peptide on the surface of silica NTCs and using a multivalent avidity approach to enhance targeting efficiency to tumor vasculature.

Prior work with SPIO-loaded polymeric micelles. With the clustering of SPIO particles in mind, Gao and coworkers successfully developed cRGD-encoded, SPIO-loaded polymer micelles. In a prior system, 9 nm SPIO nanoparticles were encapsulated (6.7 wt %) into cRGD-encoded (16%) and non-cRGD (0%) PEG-PCL micelles. In cell uptake experiments, tumor SLK endothelial cells (6×10⁶) with αᵥβ₃ over-expression were co-incubated with SPIO micelles at the final iron concentrations of 0, 25, and 75 μg/mL. MR images were collected with conventional T2-weighted spin echo acquisition parameters (TR=5000 ms, TE=80 ms). MR signal intensity of SLK cells incubated with cRGD-encoded and non-cRGD micelles. Three major conclusions can be drawn from these experiments: (1) cRGD-encoded micelles led to significant reduction of MR signal amplitude over non-cRGD micelles, primarily due to the increased micelle uptake in SLK cells; (2) non-specific uptake of non-cRGD micelles is relatively small with minimal change in MR intensity over the Fe concentration range from 0-75 μg/mL; (3) MR detection is highly sensitive in imaging tumor cells (6×10⁶ SLK cells at 3×10⁷ cells/mL) with specific αᵥβ₃-mediated uptake of Fe₃O₄ micelles. At 75 Fe μg/mL, MR amplitude decreased from 680 for non-cRGD micelles to 450 for cRGD-encoded micelles. In summary, the above preliminary data demonstrate the feasibility in producing biologically specific, highly sensitive MR molecular probes based on the micelle construct and design.

To evaluate the in vivo imaging efficacy of these polymeric micelles, prior work by one of the inventors and coworkers performed preliminary animal studies of cRGD-encoded, SPIO-loaded micelles in tumor-bearing mice. Micelle (40 nm in diameter, 12% SPIO loading, 5% cRGD density, 2 mg Fe/kg) solutions were injected into the tail vein of mice bearing breast tumors and imaged with T2-weighted sequences at 0, 4, 24 and 72 hours after micelle injection. By 72 hours, the peripheral region of the tumor had darkened noticeably, indicating accumulation of SPIO-containing micelles in that region. Meanwhile, significant micelle uptake in liver tissues was observed (data not shown). The data demonstrated the feasibility of cRGD-encoded, SPIO-loaded micelles for non-invasive detection of micelle targeting in tumor tissues. However, better nanoparticle design is necessary to increase the targeting specificity and imaging sensitivity in tumors over other healthy tissues and organs.

Anticipated improvement of NTC design over spherical micelles. SPIO dephases the MR signal of the water molecules in the surrounding environment and makes the distributed region dark on a T2-weighted MR image. One issue that has been seen is that at low doses, circulating iron may decrease the T1 time of the blood. Such undesirable T1 effect is also found for smaller SPIO particles (<10 nm) which has received favorable attention for its alternative bio-distribution effects. Thus, the T2* effect is compromised in these circumstances, because the shorter T1 components generally have brighter MR signal on the image. Using SPIO-loaded nanotubes, one can significantly enhance the T2* effect of SPIO particles and overcome the unfavorable T1 effect.

Compared to spherical particles, nanotubular capsules (NTCs) can achieve a much larger payload capacity. FIG. 5 demonstrates the difference in payload capacity of a nanoparticle with diameter 75 nm and wall thickness of 3 nm (theoretical payload capacity of 1.57×10⁵ nm³) and a nanotube with diameter 75 nm and a length of 300 nm (theoretical payload capacity of 1.06×10⁶ nm³). The difference in payload capacity is 9.0×10⁵ nm³ (the volume ratio is about 7 times). To accomplish the same payload in a spherical particle as in the previously described nanotube, the nanoparticle diameter must be increased to 132 nm. As the size of the particle increase, the cellular uptake can be limited.²² With a larger payload capacity, more SPIO particles could be loaded in the tube core. As mentioned previously, the clustering of SPIO particles results in a more sensitive probe and larger T2 relaxivity per Fe basis. These calculations demonstrate how the use of nanotubes as contrast agents could increase MR sensitivity while decreasing the NTC concentration required for detection.

Secondly, it is known that the proton resonance frequency may shift from its Lamor frequency, depending on how its surrounding electronic shielding is distributed. In principal, the magnitude of such a frequency shift, or the water signal dephasing in the case of MRI, is represented by the magnetic susceptibility anisotropy experienced by the proton:

$$Δ = \frac{1}{2 N} [ ( {χ}_{z z} - T r ( χ ) / 3 ) < \frac{3 {z}^{2} - {r}^{2}}{{r}^{5}} > + ( {χ}_{x x} - {χ}_{y y} ) < \frac{3 {x}^{2} - {y}^{2}}{{r}^{5}} > ] + \frac{1}{N} [ {χ}_{x y} < \frac{4 x y}{{r}^{5}} > + {χ}_{x z} < \frac{4 x z}{{r}^{5}} > + {χ}_{y z} < \frac{4 y z}{{r}^{5}} > ]$$

where χ is the magnetic susceptibility tensor, (x, y, z) is the Cartesian coordinates of observation nucleus, and r is the distance between the nucleus and the center of the magnetic tensor with χ_{zz} as the principal magnetic axis.

The large dephasing power of SPIO particles mainly comes from its super large magnetic moment and its slow “molecular” movements in solution. The key factor is the supermagnetic moments, which tend to align with the external magnetic field Bo along the z-direction. This results in a large (χ_{zz}−Tr(χ)/3) component and consequently causes a great signal dispersion on MRI. However, the random molecular thermal movements appose such an alignment and tend to decrease the dephasing power of SPIO particles. This is because the magnetic anisotropy experienced by surrounding water molecules can be partially averaged out over an NMR time scale as a result of the orientation fluctuation.

If SPIO particles are assembled orderly in a nanotube, the random thermal movements of SPIO could be restricted due to limited inner space and their interactions with tube walls. For a water molecule close to such a nanotube, it will feel a much larger effective magnetic moment in terms of (χ_{zz}−Tr(χ)/3) component, than it will when close to a single SPIO particle. The large local magnetic field gradient induced by the SPIO-load nanotubes at the tube ends and in the perpendicular surroundings will apparently have a larger power in dispersing the water signal than a single spherically-shaped SPIO particle. In addition, the molecular correlation time will be significantly lengthened for water trapped in the tube and hydrogen-bound to the tube walls, further enhancing T2 effect and decreasing T1 effect. Thus, one can expect a significantly larger water signal loss and MR image contrast enhancement.

The nanotubular design allows for additional surface to introduce functional ligands such as cRGD that can specifically recognize tumor markers on the surface of cancer cells. In addition, the cylindrical shape may minimize the non-specific uptake in liver or other healthy organs. For example, it has been shown that modified bacterial phages (e.g. M13) have shown much prolonged blood circulations (>3 days) due to its cylindrical shape that aligns with the blood flow which prevents its uptake by the RES system.^{23, 24} Therefore, the unique NTC design may exploit the maximum advantages of cylindrical shape in increasing cancer-specific targeting to tumor tissues with reduced uptake in healthy organs/tissues.

Nano test tubes. Template synthesis is employed to produce nanotubes, but also nano test tubes (tubes closed at one end) as will be discussed in this proposal.¹ The most useful aspect of template or membrane prepared nanotubes is the ability to control both tube length and diameter. Control of tube length and diameter is accomplished by anodic oxidation of aluminum metal to alumina; variations in time and voltage during the alumina growth result in templates of varying thickness and pore diameter. Home-grown alumina nano test tube templates were pioneered by Martin and coworkers and have been used to synthesize silica nano test tubes.¹ One advantage of template synthesizing nano test tubes is that they only need to be closed at one end to contain a payload. Also, in the loading process the payload does not escape out the open end as with nanotubes open at both ends. Another important feature of template synthesized silica nano test tubes is that the inner/outer tube surface can be differentially functionalized. Martin and coworkers have demonstrated that the inner and outer surface of silica nano test tubes can be functionalized with different silanes independently.²⁵ The inner and outer nano test tube surfaces have also been modified with antibodies as shown by Martin²⁶ and Lee²⁷ to demonstrate the utility of nano test tubes in antibody-antigen recognition. These results show the effectiveness of differential functionalization of silica nano test tubes in a wide range of applications.

Another important issue to address is the biocompatibility of silica both in vitro and in vivo. Silica nanotubes have been examined as gene delivery vehicles. These studies have demonstrated that the in vitro uptake of silica nanotubes 200 nm×2 μm caused less than 25% cell death while delivering their contained payload, demonstrating the biocompatibility and utility of silica nanotubes in delivery applications.²⁸ In vivo insertion of silica implants in mice in multiple applications have shown no adverse tissue effects or abnormal inflammation during ongoing treatments. These results also confirm the biocompatibility of silica materials.^{29, 30} In another study, silica particles with magnetic properties were injected in mice and monitored to determine mobility and clearance rate in non-targeting particles. The studies indicated that over a 4 week duration, particles were detected in almost all tissues including the brain, spleen, kidneys, testes, liver, and other organs with no toxic side effects or adverse effects on normal organ functions.³¹ These studies strongly support the use of silica in the design of our NTCs.

Capping of nano test tubes. Recently we have demonstrated that silica nano test tubes can be capped or corked covalently³ to form nanotubular capsules or NTCs. Sol-gel synthesis was used to form silica nano test tubes in the pores of the alumina template. A layer of silica also forms on the surface of the alumina template. Argon plasma etching removes the surface silica so that the tubes are not attached at their mouths, and the lips of the tubes can be modified. The silica nano test tubes were modified on their inner surface with an amino-silane and capped by placing the membrane bound tubes (˜73 nm diameter) in a solution of aldehyde modified latex particles (˜75 nm diameter). The aldehyde particles bound to the amine modified tubes by a covalent imine bond. The membrane was then dissolved and the tubes collected. The capped tubes survive the dissolving membrane conditions (>83% remain capped) indicating the multiple points of contact between the tube and cap increase the stability of the water unstable imine bond.

One key issue in developing this NTC contrast agent will be the loading of SPIO particles. Recently Martin and coworkers demonstrated the loading of nanowells with latex nanoparticles.⁴ Nanowells with diameters of ˜80 nm and a depth of ˜55 nm were fabricated. The surfaces of the nanowells were functionalized and charged latex particles ˜40 nm were deposited in the wells due to electrostatic attraction. In most cases more than one particle was deposited in the wells.⁴ In continuing studies, 15 nm and 20 nm gold colloids were deposited in the same type of nanowells. The gold colloids are able to fill the nanowells as we see a reduction in nanowell depth from 55 nm to less than 10 nm. This preliminary data suggests multiple 16 nm SPIO particles can be loaded within the pores of the membrane bound nano test tubes for use as contrast agents.

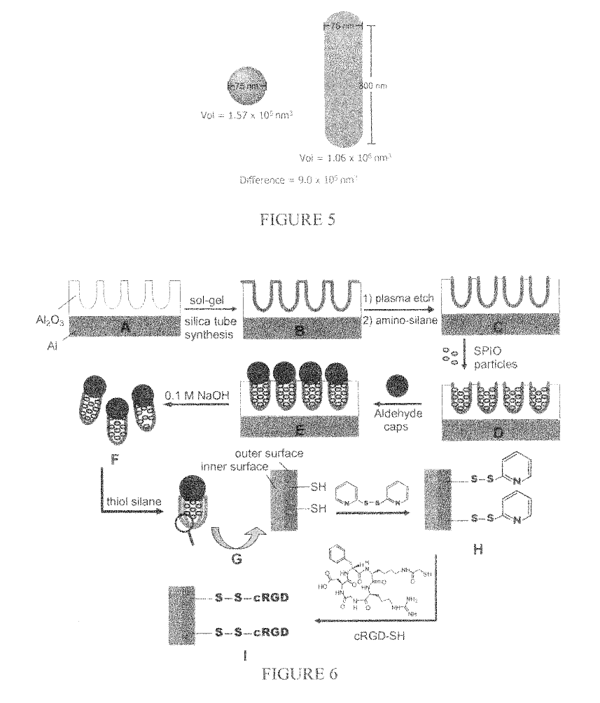

FIG. 6 is a schematic of the development and characterization of cRGD-encoded, SPIO loaded silica nanotubular capsules (cRGD-SPIO-NTCs) for in vitro studies. Template and nano test tube synthesis. Alumina nano test tube templates will be synthesized using well established procedures pioneered by Martin and coworkers.³²⁻³⁴ These templates are unique because the dimensions of the nanotube can be controlled precisely. In one example, the nanotubular templates can have diameters of 75 nm and lengths of 300 nm (6A). The NTC dimension can be tailored further depending on the outcome of biological evaluations.

The template will be modified with SiCl₄ and hydrolyzed to produce a layer-by-layer process for synthesizing silica dioxide nanotubes. In this manner, the user can control the tube wall thickness, generally 3-5 nm will be sufficient (FIG. 6B).² The surface will be etched to remove silica on the alumina template surface. The tubes can be freed from the template by submersion in acid or base for a short duration, but for our studies they will remain template-bound until they are loaded.

Loading and capping nano test tubes. While template bound, the inner surface of the silica nano test tubes will be modified with 3-aminopropyl trimethoxy silane as demonstrate previously (FIG. 6C).^{3, 25} The amine groups introduced will be used to cap the silica nano test tubes after filling them with SPIO particles. The tubes will be filled by placing the silica impregnated membrane in a solution of SPIO particles (various concentrations, FIG. 6D). The particles will fill the pores by capillary action. Further studies are underway to improve the homogeneity and loading density of SPIO in the silica nano test tubes. The tubes will be capped as described previously to prevent SPIO leakage (FIG. 6E).³

Tube functionalization with cRGD. After capping and loading the tubes will be released from the template by dissolving the template in 0.1 M NaOH (FIG. 6F). The tubes will be collected and the outer surface modified with 3-mercaptopropyl trimethoxy silane (FIG. 6G). The concentration of the thiol groups can be controlled by the addition of less/more 3-mercaptopropyl trimethoxy silane. Once the thiol layer has cured, the NTCs will be resuspended in a solution of 2,2′-dipyridyl disulfide to produce an activated disulfide on the nano test tube surface (FIG. 6H). The byproduct of the reaction is 2-pyridinethione which can be monitored by UV spectroscopy. This serves two purposes. First, the extent of thiol functionality on the NTC surface can be determined by collecting the NTCs by filtration and examining the filtrate to determine the concentration of 2-pyridinethione. The amount of 2-pyridinethione directly relates to the number of activated disulfides on the tube surface. Second, after NTC surface functionalization with the activated disulfides, cRGD-SH will be coupled to the tubes (FIG. 6I) and the extent of cRGD modification can once again be determined by the resulting 2-pyridinethione concentration in solution. Based on the different thiol surface densities, cRGD density can be controlled on the NTC surface to be 5, 10, and 20%.

Characterization of cRGD-SPIO-NTCs. The outer surface functionalization by cRGD peptides will be characterized by X-ray photoelectron spectroscopy (XPS) to quantify the N signals from the peptides. The NTC morphology, SPIO loading and capping extent will be determined by transmission electron microscopy (TEM). The magnetic properties (e.g., saturated magnetization moment) will be measured by a SQUID instrument, which will be correlated to the T2 relaxivity by a 4.7 MRI scanner.

Cell uptake of cRGD-functionalized, SPIO-loaded nanotubular capsules, c-RGD-SPIO-NTCs). SLK cells will be seeded at 125,000 cells/well in 6-well plates in 2 mL DMEM medium with 10% fetal bovine serum. After 24 hours, 1 mg of NTCs (from 3 mg/mL NTC suspension) will be added into each well and incubated at 37° C. for 1 hour. To test αᵥβ₃ specificity, we will also add a αᵥβ₃ blocking antibody (LM609) during NTC incubation with SLK cells. Then, cells will be washed, trypsinized and neutralized. After centrifugation at 1200 rpm for 5 min, cells will be re-suspended in 1 mL PBS. The total number of cells will be measured by hemocytometer. T₂-weighted MR images of cells mixed in agarose gel will be obtained. We will compare MR intensity of cells treated with different NTC concentrations. This data will provide useful in vitro comparison for future in vivo studies.

Evaluation of the in vivo imaging efficacy of cRGD-SPIO-NTCs in mice bearing breast tumor xenografts by MRI. Contrast enhancement of cRGD-functionalized, SPIO-loaded NTCs upon accumulation in tumors in vivo. Mice with MCF-7 and MB-MDA-231 breast tumor xenographs will be used for this study. The cell lines will be injected to form subcutaneous tumors. Tumors will be allowed to grow to sufficient size to induce angiogenic vessels that over express αᵥβ₃ integrins. In this study, tumor xenographs of comparable sizes will be used to evaluate the effect of NTC doses on contrast enhancement of the NTCs. cRGD-functionalized, SPIO-loaded NTCs of different cRGD densities will be injected at different doses via the mouse tail vein. For each group of cRGD density, 3 doses of NTC solutions will be tested. All experiments will be conducted in triplicate to ensure the reproducibility of the data. T₂-weighted MR images of tumors will be obtained using a spin-echo pulse sequence at 4.7T. Image processing will be carried out using ImageJ (NIH).

An optimal NTC dose may exist to provide the highest tumor contrast. A fine line exists between NTC doses. Higher NTC doses may lead to a larger amount of NTCs accumulating in the breast tumors to enhance MR detection. Too high doses may increase the non-specific background contrast after saturating the αᵥβ₃ binding sites on tumor endothelial cells. Initially, we will evaluate the optimal NTC dosage using cRGD-SPIO-NTCs by examining tumor contrast over the surrounding muscle tissue 2 hours after injection. The ratios of signal intensities will be plotted as a function of injected NTC dose (in Fe mg/kg). An optimal dose will be determined for cRGD-SPIO-NTCs for subsequent studies.

Evaluation of αᵥβ₃ specificity in angiogenesis imaging in breast tumors. Based on the best NTC formulations and doses established above, we will evaluate the time-dependent accumulation of NTCs in breast tumors. Twelve tumor-bearing mice will be divided into three treatment regimen groups and receive either: (1) cRGD-SPIO-NTCs (n=4), (2) cRGD free SPIO-NTCs (n=4), and (3) co-injection of free cRGD ligand and cRGD-SPIO-NTCs (i.e. competition group, n=4). The cRGD-free control group allows the evaluation of NTC accumulation in breast tumors due to “passive targeting” as a result of leaky tumor vasculature.

The MR images will be analyzed to determine the relative NTC concentrations within tumors as a function of time. A tumor contrast map will be generated by subtracting the pre-contrast image from the post-contrast image. On each imaging slice, a region of interest (ROI) will be outlined to cover the signal decreasing lesions based on the color-coded contrast map 2 hours after injection. Signal-time curves will be obtained from every imaging slice containing the lesion. Curves will also be measured from two edge slices (the beginning and ending slice), but if partial volume effects are noted, the data will be discarded. A mean signal intensity-time curve for each lesion will be produced by averaging the time courses measured from the remaining slices. Based on the signal intensity-time curves, we will determine the time that potentially reaches “steady state” and its duration, and magnitude of signal intensity change for different NTC formulations. Contrast changes in the other organs (e.g. liver and kidney) will also be examined to evaluate non-specific uptake. After MR imaging, breast tumors will be resected for histology and immunohistochemistry to verify tumor pathology and access microvacularity and angiogenesis following published procedures.^{35, 36} A mean peak vessel count is determined for each tumor by averaging the counts from the three hypervascular areas as a measure of microvascular density (MVD). This value will be correlated to the MR data to evaluate the αᵥβ₃ specificity in angiogenesis imaging in breast tumors.

Statistical Analysis. Various methods will be employed to provide data analysis and comparison on MR and histology images. Pearson's linear regression will be used to determine the degree of correlation between the MRI parameters (e.g. ROI mean value) and histology data (MVD) at different time points for different NTC formulations. The linear correlation coefficient, r, and the 95% confidence intervals will be reported. For paired comparisons between groups, we will use Student's t-test and P-values to statistically evaluate the significance between the NTC groups.

Potential Outcomes. These studies will help establish a novel MRI molecular probe to image breast cancer cells at the onset of tumor growth. Early detection by targeting αᵥβ₃ integrins on tumor vasculature may allow patients to obtain more effective treatments with fewer resulting side effects. The use of NTCs in MR imaging will provide a less invasive procedural option for patients with improved accuracy over conventional methods such as mammography. The ultra-sensitive probe design is advantageous because the concentration of NTCs required for detection will be greatly reduced, resulting in the ability to monitor molecular processes in vivo without disrupting any natural processes and provide a new method of detection before the onset of symptoms. Although the focus of this proposal was on breast cancer detection, the technology developed can be focused on lung and prostate cancer also. A panel of 50 different lung cancer lines to further develop and validate NTC design can be used.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Gasparac, R.; Kohli, P.; Paulino, M. O. M.; Trofin, L.; Martin, C. R., Template synthesis of nano test tubes. Nano Letters 2004, 4, (3), 513-516.
2. Kovtyukhova, N. I.; Mallouk, T. E.; Mayer, T. S., Templated surface sol-gel synthesis of SiO2 nanotubes and SiO2-insulated metal nanowires. Advanced Materials (Weinheim, Germany) 2003, 15, (10), 780-785.
3. Hillebrenner, H.; Buyukserin, F.; Kang, M.; Mota, M. O.; Stewart, J. D.; Martin, C. R., Corking Nano Test Tubes by Chemical Self-Assembly. Journal of the American Chemical Society 2006, 128, (13), 4236-4237.
4. Kang, M.; Yu, S.; Li, N.; Martin, C. R., Nanowell-Array Surfaces Prepared by Argon Plasma Etching through a Nanopore Alumina Mask. Langmuir 2005, 21, (18), 8429-8438.
5. Jemal, A.; Murray, T.; Ward, E.; Samuels, A.; Tiwari Ram, C.; Ghafoor, A.; Feuer Eric, J.; Thun Michael, J., Cancer statistics, 2005. CA: a cancer journal for clinicians 2005, 55, (1), 10-30.
6. Huang, W.; Fisher Paul, R.; Dulaimy, K.; Tudorica Luminita, A.; O'Hea, B.; Button Terry, M., Detection of breast malignancy: diagnostic MR protocol for improved specificity. Radiology 2004, 232, (2), 585-91.
7. Axelsson, K.; Ljung, B. M.; Moore, D. H., 2nd; Thor, A. D.; Chew, K. L.; Edgerton, S. M.; Smith, H. S.; Mayall, B. H., Tumor angiogenesis as a prognostic assay for invasive ductal breast carcinoma. Journal of the National Cancer Institute 1995, 87, (13), 997-1008.
8. Weidner, N., Intratumor microvessel density as a prognostic factor in cancer. The American journal of pathology 1995, 147, (1), 9-19.
9. Brian, M.; Jesberger John, A.; Lewin Jonathan, S.; Hillenbrand Claudia, M.; Duerk Jeffrey, L., Determining and optimizing the precision of quantitative measurements of perfusion from dynamic contrast enhanced MRI. Journal of magnetic resonance imaging: JMRI 2003, 18, (5), 575-84.
10. Orel, S. G.; Schnall, M. D., MR imaging of the breast for the detection, diagnosis, and staging of breast cancer. Radiology 2001, 220, (1), 13-30.
11. Su, M. Y.; Jao, J. C.; Nalcioglu, O., Measurement of vascular volume fraction and blood-tissue permeability constants with a pharmacokinetic model: studies in rat muscle tumors with dynamic Gd-DTPA enhanced MRI. Magnetic resonance in medicine: official journal of the Society of Magnetic Resonance in Medicine/Society of Magnetic Resonance in Medicine 1994, 32, (6), 714-24.
12. Su, M. Y.; Wang, Z.; Carpenter, P. M.; Lao, X.; Muhler, A.; Nalcioglu, O., Characterization of N-ethyl-N-nitrosourea-induced malignant and benign breast tumors in rats by using three MR contrast agents. Journal of magnetic resonance imaging: JMRI 1999, 9, (2), 177-86.
13. Su, M.-Y.; Cheung, Y.-C.; Fruehauf John, P.; Yu, H.; Nalcioglu, O.; Mechetner, E.; Kyshtoobayeva, A.; Chen, S.-C.; Hsueh, S.; McLaren Christine, E.; Wan, Y.-L., Correlation of dynamic contrast enhancement MRI parameters with microvessel density and VEGF for assessment of angiogenesis in breast cancer. Journal of magnetic resonance imaging: JMRI 2003, 18, (4), 467-77.
14. Tofts, P. S.; Brix, G.; Buckley, D. L.; Evelhoch, J. L.; Henderson, E.; Knopp, M. V.; Larsson, H. B.; Lee, T. Y.; Mayr, N. A.; Parker, G. J.; Port, R. E.; Taylor, J.; Weisskoff, R. M., Estimating kinetic parameters from dynamic contrast-enhanced T(1)-weighted MRI of a diffusable tracer: standardized quantities and symbols. Journal of magnetic resonance imaging JMRI 1999, 10, (3), 223-32.
15. Wang, Y.; Hussain, S.; Krestin, G., Supraparamagnetic iron oxide contrast agents: physiochemical characteristics and applications in MR imaging. European Radiology 2001, 2001, (11), 2319-2331.
16. Nasongkla, N.; Shuai, X.; Ai, H.; Weinberg, B. D.; Pink, J.; Boothman, D. A.; Gao, J., Bioorganic chemistry: cRGD-functionalized polymer micelles for targeted doxorubicin delivery. Angewandte Chemie, International Edition 2004, 43, (46), 6323-6327.
17. Nie, S.; Kim, G. J., Targeted Cancer Nanotherapy. Materials Today 2005, 8, (8), 28-33.
18. Brooks, P. C.; Montgomery, A. M.; Rosenfeld, M.; Reisfeld, R. A.; Hu, T.; Klier, G.; Cheresh, D. A., Integrin alpha v beta 3 antagonists promote tumor regression by inducing apoptosis of angiogenic blood vessels. Cell 1994, 79, (7), 1157-64.
19. Xiong, J.-P.; Stehle, T.; Diefenbach, B.; Zhang, R.; Dunker, R.; Scott, D. L.; Joachimiak, A.; Goodman, S. L.; Arnaout, M. A., Crystal structure of the extracellular segment of integrin aVb3. Science (Washington, D.C., United States) 2001, 294, (5541), 339-345.
20. Xiong, J.-P.; Stehle, T.; Zhang, R.; Joachimiak, A.; Frech, M.; Goodman, S. L.; Arnaout, M. A., Crystal structure of the extracellular segment of integrin avb3 in complex with an Arg-Gly-Asp ligand. Science (Washington, D.C., United States) 2002, 296, (5565), 151-155.
21. Hood, J. D.; Bednarski, M.; Frausto, R.; Guccione, S.; Reisfeld, R. A.; Xiang, R.; Cheresh, D. A., Tumor regression by targeted gene delivery to the neovasculature. Science (Washington, D.C., United States) 2002, 296, (5577), 2404-2407.
22. Allen, T. M.; Cullis, P. R., Drug Delivery Systems: Entering the Mainstream. Science (Washington, D.C., United States) 2004, 303, (5665), 1818-1822.
23. Merril, C. R.; Biswas, B.; Carlton, R.; Jensen, N. C.; Creed, G. J.; Zullo, S.; Adhya, S., Long-circulating bacteriophage as antibacterial agents. Proceedings of the National Academy of Sciences of the United States of America 1996, 93, (8), 3188-92.
24. Pasqualini, R.; Koivunen, E.; Ruoslahti, E., av Integrins as receptors for tumor targeting by circulating ligands. Nature Biotechnology 1997, 15, (6), 542-546.
25. Mitchell, D. T.; Lee, S. B.; Trofin, L.; Li, N.; Nevanen, T. K.; Soederlund, H.; Martin, C. R., Smart nanotubes for bioseparations and biocatalysis. Journal of the American Chemical Society 2002, 124, (40), 11864-11865.
26. Lee, S. B.; Mitchell, D. T.; Trofin, L.; Nevanen, T. K.; Soederlund, H.; Martin, C. R., Antibody-based bio-nanotube membranes for enantiomeric drug separations. Science (Washington, D.C., United States) 2002, 296, (5576), 2198-2200.
27. Son, S. J.; Reichel, J.; He, B.; Schuchman, M.; Lee, S. B., Magnetic nanotubes for magnetic-field-assisted bioseparation, biointeraction, and drug delivery. Journal of the American Chemical Society 2005, 127, (20), 7316-7317.
28. Chen, C.-C.; Liu, Y.-C.; Wu, C.-H.; Yeh, C.-C.; Su, M.-T.; Wu, Y.-C., Preparation of Fluorescent Silica Nanotubes and their Application in Gene Delivery. Advanced Materials (Weinheim, Germany) 2005, 17, (4), 404-407.
29. Barbe, C.; Bartlett, J.; Kong, L.; Finnie, K.; Lin, H. Q.; Larkin, M.; Calleja, S.; Bush, A.; Calleja, G., Silica particles: A novel drug-delivery system. Advanced Materials (Weinheim, Germany) 2004, 16, (21), 1959-1966.
30. Radin, S.; El-Bassyouni, G.; Vresilovic, E. J.; Schepers, E.; Ducheyne, P., In vivo tissue response to resorbable silica xerogels as controlled-release materials. Biomaterials 2004, 26, (9), 1043-1052.
31. Kim, J. S.; Yoon, T.-J.; Yu, K. N.; Kim, B. G.; Park, S. J.; Kim, H. W.; Lee, K. H.; Park, S. B.; Lee, J.-K.; Cho, M. H., Toxicity and Tissue Distribution of Magnetic Nanoparticles in Mice. Toxicological Sciences 2006, 89, (1), 338-347.
32. Masuda, H.; Hasegwa, F.; Ono, S., Self-ordering of cell arrangement of anodic porous alumina formed in sulfuric acid solution. Journal of the Electrochemical Society 1997, 144, (5), L127-L130.
33. Masuda, H.; Fukuda, K., Ordered metal nanohole arrays made by a two-step replication of honeycomb structures of anodic alumina. Science (Washington, D.C.) 1995, 268, (5216), 1466-68.
34. Masuda, H.; Satoh, M., Fabrication of gold nanodot array using anodic porous alumina as an evaporation mask. Japanese Journal of Applied Physics, Part 2: Letters 1996, 35, (1B), L126-L129.
35. Sipkins, D. A.; Cheresh, D. A.; Kazemi, M. R.; Nevin, L. M.; Bednarski, M. D.; Li, K. C. P., Detection of tumor angiogenesis in vivo by avb3-targeted magnetic resonance imaging. Nature Medicine (New York) 1998, 4, (5), 623-626.
36. Patrick, M.; Caruthers Shelton, D.; Kassner, A.; Harris Thomas, D.; Chinen Lori, K.; Allen John, S.; Lacy Elizabeth, K.; Zhang, H.; Robertson, J. D.; Wickline Samuel, A.; Lanza Gregory, M., Molecular imaging of angiogenesis in nascent Vx-2 rabbit tumors using a novel alpha(nu)beta3-targeted nanoparticle and 1.5 tesla magnetic resonance imaging. Cancer research 2003, 63, (18), 5838-43.

What is claimed is:

1. An MRI contrast agent comprising:
a generally nanotubular carrier; and
an MRI contrast agent disposed within the carrier.

2. The MRI contrast agent of claim 1, wherein the carrier is biocompatible, biodegradable or both.

3. The MRI contrast agent of claim 1, wherein the carrier comprises one or two open ends.

4. The MRI contrast agent of claim 1, wherein the carrier comprises one or two open ends and one or both are capped.

5. The MRI contrast agent of claim 1, carrier comprises a biodegradable polymer selected from polysaccharides, cellulose, chitosan, carboxymethylated cellulose, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides, polyalkyl(cyano)acrylates, poly(trimethylene carbonate) and copolymers, poly (ε-caprolactone) homopolymers and copolymers, polyhydroxybutyrate and polyhydroxyvalerate, poly(ester)urethanes and copolymers, polymethyl-methacrylate and combinations thereof.

6. The MRI contrast agent of claim 1, wherein carrier is selected from polyglutamic or polyaspartic acid derivatives and their copolymers with other amino-acids.

7. The MRI contrast agent of claim 1, wherein the contrast agent comprises superparamagnetic iron oxide nanoparticles.

8. The MRI contrast agent of claim 1, wherein the contrast agent comprises a superparamagnetic iron oxide selected from the compositions of MFe₂O₄, wherein M=Fe, Co, Ni, Zn, Mg, Mn divalent metal ions).

9. The MRI contrast agent of claim 1, wherein the contrast agent comprises a hydrophilic MRI contrast agent.

10. The MRI contrast agent of claim 1, wherein the carrier comprises a silica tubule and the contrast agent comprises superparamagnetic iron oxide nanoparticles and the contrast agent is detectable at a concentration of less that 10 pM.

11. The MRI contrast agent of claim 1, wherein the carrier is functionalized.

12. The MRI contrast agent of claim 1, wherein the carrier is functionalized and a targeting ligand is bound to the carrier.

13. The MRI contrast agent of claim 1, wherein the carrier is functionalized with amines, carboxylic acids, thiols, aldehydes and combinations thereof.

14. The MRI contrast agent of claim 1, wherein the carrier is functionalized with a cross-linking agent selected from glutaraldehydes, diamines, and disulfides and combinations thereof.

15. The MRI contrast agent of claim 1, wherein the carrier is functionalized and a targeting ligand is selected from aptamers, peptides, small organic molecules, antibodies, proteins, folic acid, oligopeptides and oligosaccharides.

16. The MRI contrast agent of claim 1, wherein the carrier comprises a biocompatible inorganic tubule selected from iron oxide, titanium dioxide, silicon oxide or combinations thereof.

17. A method for making an MRI contrast agent comprising:
forming a nanotubular carrier; and
loading the nanotubular carrier with an MRI contrast agent.

18. The method of claim 17, wherein the carrier is biocompatible, biodegradable or both.

19. The method of claim 17, wherein the carrier comprises one or two open ends.

20. The method of claim 17, wherein the carrier comprises one or two open ends and one or both are capped.

21. The method of claim 17, carrier comprises a biodegradable polymer selected from polysaccharides, cellulose, chitosan, carboxymethylated cellulose, polyamino-acids, polylactides and polyglycolides and their copolymers, copolymers of lactides and lactones, polypeptides, poly-(ortho)esters, polydioxanone, poly-β-aminoketones, polyphosphazenes, polyanhydrides, polyalkyl(cyano)acrylates, poly(trimethylene carbonate) and copolymers, poly (ε-caprolactone) homopolymers and copolymers, polyhydroxybutyrate and polyhydroxyvalerate, poly(ester)urethanes and copolymers, polymethyl-methacrylate and combinations thereof.

22. The method of claim 17, wherein carrier is selected from polyglutamic or polyaspartic acid derivatives and their copolymers with other amino-acids.

23. The method of claim 17, wherein the contrast agent comprises superparamagnetic iron oxide nanoparticles.

24. The method of claim 17, wherein the contrast agent comprises a superparamagnetic iron oxide selected from the compositions of MFe₂O₄, where M=Fe, Co, Ni, Zn, Mg, Mn divalent metal ions).

25. The method of claim 17, wherein the carrier comprises a silica tubule and the contrast agent comprises superparamagnetic iron oxide nanoparticles and the contrast agent is detectable at a concentration of less that 10 pM.

26. The method of claim 17, wherein the carrier is functionalized.

27. The method of claim 17, wherein the carrier is functionalized and a targeting ligand is bound to the carrier.

28. The method of claim 17, wherein the carrier is functionalized with amines, carboxylic acids, thiols, aldehydes and combinations thereof.

29. The method of claim 17, wherein the carrier is functionalized with a cross-linking agent selected from glutaraldehydes, diamines, and disulfides and combinations thereof.

30. The method of claim 17, wherein the carrier is functionalized and a targeting ligand is selected from aptamers, peptides, small organic molecules, antibodies, proteins, folic acid, oligopeptides and oligosaccharides.

31. The method of claim 17, wherein the carrier comprises a biocompatible inorganic tubule selected from iron oxide, titanium dioxide, silicon oxide or combinations thereof.

32. The method of claim 17, wherein the contrast agent comprises a hydrophilic MRI contrast agent.

33. A method for assessing tissue in a patient using a magnetic resonance imaging (MRI) apparatus, the method comprising:
injecting into the patient a generally tubular nanocarrier comprising an MRI contrast agent within the nanocarrier.

34. An MRI contrast agent comprising:
a nanotubular carrier;
an MRI contrast agent loaded into the carrier; and
a targeting ligand bound to the carrier.

35. A method for making an MRI contrast agent comprising:
forming a nanotubular carrier;
loading the nanotubular carrier with an MRI contrast agent; and
functionalizing the surface of the carrier a targeting ligand.
